(19) United States
(12) Patent Application Publication
(10) Pub. No.: US 2012/0328566 A9
(43) Pub. Date: Dec. 27, 2012

(54) ESTROGEN RECEPTOR LIGAND TREATMENT FOR NEURODEGENERATIVE DISEASES

(76) Inventor: Rhonda R Voskuhl, Los Angeles, CA (US)

(21) Appl. No.: 12/998,463

(22) PCT Filed: Oct. 31, 2008
(86) PCT No.: PCT/US2008/012353

§ 371 Date: Jun. 7, 2011

Related U.S. Application Data
(63) Continuation-in-part of application No. 11/151,040, filed on Jun. 13, 2005.
Application No. 11/151,040 is a continuation of application No. 10/131,834, filed on Apr. 24, 2002, now Pat. No. 6,936,599.
(60) Provisional application No. 60/286,842, filed on Apr. 25, 2001.

(51) Int. Cl. A61K 38/21; A61P 25/16; A61P 25/28; A61P 25/00

(52) U.S. Cl. 424/85.6

(57) ABSTRACT

The present invention relates to treatment of neurological diseases such as multiple sclerosis (MS) and Alzheimer's disease, using an estrogen receptor beta (ERβ) ligand in combination with a standard, anti-inflammatory agent.

This invention was made with Government support under Grant No. NS454443 awarded by the National Institute of Health. The government has certain rights in this invention.

### BACKGROUND OF THE INVENTION

1. Field of the Invention

This invention relates generally to a novel treatment to prevent neurodegeneration in the central nervous system due to diseases such as multiple sclerosis (MS), Alzheimer's disease, Parkinson's disease, spinal cord injury, stroke, etc. More specifically, the present invention relates to a treatment comprising a combination of an estrogen receptor ligand with a secondary agent, such as an immunotherapeutic compound.

2. General Background

There are no neuroprotective drugs that can be taken for long durations of time without significant side effects. Estrogens, as well as the use of estrogen receptor (ER) alpha ligand treatments, have been studied in disease and injury models and in humans. Estrogen, and estrogen receptor alpha ligand treatments, are effective in some disease and injury models. For example, they are both anti-inflammatory and neuroprotective in experimental autoimmune encephalomyelitis (EAE), the animal model for multiple sclerosis (MS) and there is a dose response whereby higher levels are more protective. However, in humans, treatment with estrogens or ER alpha ligands may not be tolerable due to the induction of breast cancer and uterine cancer, which are mediated by estrogen receptor alpha in the breast and uterus, respectively. One must always consider the risk:benefit ratio of any estrogen treatment when considering its use in neurodegenerative diseases. Estrogens in the form of hormone replacement therapy have been associated with side effects and therefore are not recommended for use in healthy menopausal women. While the risk:benefit ratio in debilitating neurodegenerative diseases is clearly different than the risk:benefit ratio in healthy individuals, optimizing efficacy and minimizing toxicity, remains the goal. Hence, determining which estrogen receptor mediates the neuroprotective effect of estrogen treatment is of central importance.

In contrast, estrogen receptor beta (ERβ) is not associated with breast or uterine cancer. Thus, estrogen receptor beta ligands may be used for long durations and/or for high risk patients who could not otherwise tolerate estrogen or estrogen receptor alpha ligand treatment.

One estrogen, estradiol, and estrogen receptor alpha ligands agonist have been shown to be both anti-inflammatory and neuroprotective in the EAE model. They ameliorate EAE symptomology immediately after the disease is induced. In contrast, estrogen receptor beta ligand treatment is not anti-inflammatory, but has been shown for the first time to be neuroprotective. This mechanism is thought to explain why ER beta ligand treatment does not work at EAE onset, but does work later to promote recovery or delay EAE progression.

There are currently no purely neuroprotective treatments for MS. Thus, for diseases such as MS which have both an inflammatory and a neurodegenerative component, estrogen receptor beta ligands may be useful. For diseases that do not appear to have an inflammatory component, but only a neurodegenerative component, then the estrogen receptor beta ligand treatment may also be useful. Notably, the role of inflammation in Alzheimer's disease, Parkinson's disease, brain or spinal cord injury and stroke are primarily purely neurodegenerative diseases or injuries, but there may be a minor inflammatory component. To date, for Alzheimer's disease, for example, there are only treatments that can be used in short term duration. Thus, alternative treatments are desirable.

Despite the fact ERβ has been shown to be expressed widely in the CNS in adult mice, in most neurological disease models, the protective effect of estrogen treatment has been shown to be mediated through ERα and has been associated with anti-inflammatory effects. Whether neuroprotective effects could be observed in the absence of an anti-inflammatory effect remained unknown, with a recent study suggesting that an anti-inflammatory effect was necessary to observe neuroprotection in stroke. Importantly, data showing a protective effect using the ERβ ligand, diarylpropionitrile (DPN), in EAE were particularly surprising given that another ERβ ligand (WAY-202041) was shown to have no effect in EAE.

Presently, the only previously described neuroprotective agent for EAE, which did not decrease CNS inflammation, were blockers of glutamate receptors. These treatments resulted in a modest reduction in neurologic impairment and the effect was lost after cessation of treatment. Glutamate blockers are currently used in amyotrophic lateral sclerosis (ALS) and Alzheimer's disease with modest success. In MS, brain atrophy on MRI has been detected at the early stages of disease, thus a neuroprotective agent would need to be started relatively early, generally at ages 20-40 years, and continued for decades. Since glutamate is needed for normal neuronal plasticity and memory, treatment of relatively young individuals with glutamate blockers for decades may be associated with significant toxicity. Hence, the identification of an alternative neuroprotective agent represents an important advance in preclinical drug development in MS and other chronic neurodegenerative diseases or injuries.

### INVENTION SUMMARY

The present invention is directed to a treatment to prevent neurodegeneration in the central nervous system due to diseases such as MS, Parkinson's disease, cerebellar ataxia, Down's Syndrome, epilepsy, strokes, Alzheimer's disease, as well as brain and/or spinal cord (CNS) injury.

In accordance with one embodiment of the present invention, a method for treating the symptoms of a neurodegenerative disease or CNS injury in a mammal is provided, the method comprising the steps of administering to the mammal a primary agent being an estrogen receptor ligand and a secondary agent being an immunotherapeutic compound.

In accordance with another embodiment of the present invention, the invention comprises the use of a primary agent comprising an estrogen receptor beta ligand for a neuroprotective effect. In one embodiment ERβ may be used to delay the onset or progression of disease or injury after the acute phase and/or decrease the ameliorate clinical symptoms of neurodegenerative diseases or injury, including multiple sclerosis. In one embodiment, the immunotherapeutic compound comprises interferon beta (IFN-β). At least one advantage of this invention is to reduce the dosage of β interferon to patients, which causes flu-like symptoms.

In accordance with yet another embodiment the present invention relates to Use of at least one primary therapeutically active agent, the primary therapeutically active agent being an estrogen receptor ligand, in combination with a secondary active agent, the secondary active agent being beta interferon for the manufacture of a medicament for the therapeutic treatment of a neurodegenerative disease in a mammal.

The above described and many other features and attendant advantages of the present invention will become apparent from a consideration of the following detailed description when considered in conjunction with the accompanying drawings.

### BRIEF DESCRIPTION OF THE DRAWINGS

FIG. 1 is a bar graph depicting doses of the ERα and ERβ ligands relative to a biological response on a positive control tissue, the uterus.

FIGS. 2A-C are graphs depicting treatment with (A) ERα, and (B) ERβ selective ligands in wild-type and (C) knock out animals relative to mean clinical scores in the EAE model.

FIGS. 3A-C are bar graphs depicting treatment with ERα and ERβ selective ligands relative to the systemic immune response (TNF-alpha, IFN-gamma, IL-6 and IL-5 all pg/ml).

FIGS. 4A-D are microphotographs showing inflammation in early (A) and late (B) EAE progression, and are bar graphs depicting cell density following treatment with an ERα ligand and ERβ ligand, early (C) and late (D) in spinal cords of mice with EAE.

FIGS. 5A-D are photomicrographs showing myelin in early (A) and late (B) EAE progression, and are bar graphs depicting myelin density following treatment with an ERα ligand and an ERβ ligand, early (C) and late (D) in white matter of spinal cords of mice with EAE.

FIGS. 6A-D are photomicrographs showing axonal staining in early (A) and late (B) EAE progression and are bar graphs depicting axonal densities following treatment with an ERα ligand and an ERβ ligand, early (C) and late (D) in mice with EAE.

FIGS. 7A-D are photomicrographs showing neuronal staining in early (A) and late (B) EAE progression and are bar graphs depicting neuronal survival with an ERα ligand and an ERβ ligand, early (C) and late (D) neuronal staining in gray matter of spinal cords of mice with EAE.

FIGS. 8A and B are graphs depicting time on rotorod test following treatment with an ERβ ligand in wild type (A) or knock out mice (B) showing recovery of motor function late during EAE following treatment.

FIGS. 9A and B depict photographs of spinal cords with H&E and DAPI staining after treatment with an ERα ligand, ERβ ligand, early (A) and late (B) in EAE progression, and reduced inflammation in spinal cords of mice with EAE: H&E and DAPI staining.

FIGS. 10A and B are photomicrographs showing neuropathology in grey and white matter, and C-E are bar graphs depicting protection from neuropathology during EAE is dependent upon ERβ as measured in (c) white matter cell density; (D) myelin density; (E) axon number; and (F) neuron number after ERβ ligand treatment in ER beta knock out mice.

FIG. 11 is a graph depicting EAE severity score after a combination treatment with IFNβ and an ERβ ligand relative to a vehicle, or IFNB alone. The combination reduced the severity of disability in EAE.

### DETAILED DESCRIPTION

This description is not to be taken in a limiting sense, but is made merely for the purpose of illustrating the general principles of the invention. The section titles and overall organization of the present detailed description are for the purpose of convenience only and are not intended to limit the present invention.

Generally, the invention involves a method of treating a mammal exhibiting clinical symptoms of an autoimmune or neurodegenerative disease comprising administering a primary agent being an estrogen receptor ligand and a secondary agent being an immunotherapeutic compound. The treatment is aimed at providing a protective effect after the acute phase, reducing the degree of symptomology and/or progression of an autoimmune or neurodegenerative disease.

The beneficial effect of treatment can be evidenced by a protective effect on the progression of disease symptomology after the acute phase, a reduction in the severity of some or all of the clinical symptoms, or an improvement in the overall health.

For example, patients who have clinical symptoms of an autoimmune and/or neurodegenerative disease often suffer from some or all of the following symptoms: worsening of pre-existing symptoms (such as joint pain in rheumatoid arthritis), the appearance of new symptoms (new joints affected in rheumatoid arthritis) or increased generalized weakness and fatigue. MS patients in particular suffer from the following symptoms: weakness, numbness, tingling, loss of vision, memory difficulty and extreme fatigue. Thus, an amelioration of disease in MS would include a reduction in the frequency or severity of onset of weakness, numbness, tingling, loss of vision, memory difficulty and extreme fatigue. On imaging of the brain (MRI) amelioration or reduced progression of disease would be evidenced by a decrease in the number or volume of gadolinium enhancing lesions, a stabilization or slowing of the accumulation of T2 lesions and/or a slowing in the rate of atrophy formation. Immunologically, an increase in Th2 cytokines (such as IL-10) a decrease in Th1 cytokines (such as interferon gamma) would be associated with disease amelioration.

Patients may also express criteria indicating they are at risk for developing autoimmune diseases. These patients may be preventatively treated to delay the onset of clinical symptomology. More specifically, patients who present initially with clinically isolated syndromes (CIS) may be treated using the treatment paradigm outlined in this invention. These patients have had at least one clinical event consistent with MS, but have not met full criteria for MS diagnosis since the definite diagnosis requires more than one clinical event at another time (McDonald et al., 2001). Treatment of the present invention would be advantageous at least in providing a protective effect after the acute phase of clinically definite MS.

PRIMARY AGENT. The primary agent useful in this invention is an estrogen receptor β agonists. These agonists may be steroidal or non-steroidal agents which bind to and/or cause a change in activity or binding of the estrogen receptor β. For example, specific agonists of ERβ may be useful in this invention (Fritzmeier, et al.). In one embodiment, an ER beta agonist useful in this invention is the non-steroidal analog diarylpropionitrile (DPN). Additionally, analogues of ERβ agonists that are more selective for ERβ than ERα, which are know to those skilled in the art, may also be useful in the present invention. For example, ER beta agonists which are analogs to DPN are known in the art (Harrington, W R et al., “Activities of estrogen receptor alpha- and beta-selective ligands at diverse estrogen responsive gene sites mediating transactivation or transrepression,” Molecular and Cellular Endocrinology, 29 Aug. 2003, vol. 206(1-2), pp. 12-22; Meyers, M J et al., “Estrogen receptor-beta potency-selective ligands: structure-activity relationship studies of diarylpropionitriles and their acetylene and polar analogues,” Journal of Medicinal Chemistry, 22 Nov. 2001, vol. 44(24), pp. 4230-4251). Doses of these agonists may be titrated to achieve an effect on disease by methodologies known to those skilled in the art of receptor pharmacology.

SECONDARY ACTIVE AGENTS. Any one or a combination of secondary active agents may be included in combination with the primary agent. Alternatively, any one or a combination of secondary active agents may be administered independently of the primary agent, but concurrent in time for exposure to at least two agents for the treatment of the autoimmune or neurodegenerative immunological disease.

The secondary agents are preferably immunotherapeutic agents, which act synergistically with the primary agent to diminish the symptomology of the neurodegenerative disease. Secondary active agents may be selected to enhance the effect of the primary agent, or effect a different system than that effected by the primary agent.

The secondary agent may be selected from the group comprising β-interferon compounds. Examples include as β-interferon (Avonex® (interferon-beta 1a), Rebiff® (by Serono); Biogen, Betaseron® (interferon-beta 1b; Berlex, Schering).

Optionally, the following tertiary agents may be used: glatiramer acetate (Copaxone®; Teva), antineoplastics (such as mitoxantrone; Novatrone® Lederle Labs), human monoclonal antibodies (such as natalizumab; Antegren® Elan Corp. and Biogen Inc.), immonusuppressants (such as mycophenolate mofetil; CellCept® Hoffman-LaRoche Inc.), paclitaxel (Taxol®; Bristol-Meyers Oncology), cyclosporine (such as cyclosporin A), corticosteroids (glucocorticoids, such as prednisone and methyl prednisone), azathioprine, cyclophosphamide, methotrexate, cladribine, 4-aminopyridine and tizanidine

In yet other embodiments, additional agents may be added to the combination at a therapeutically effective amount. Preferably the additional agent may be administered at a lower dose due to the synergistic effect with the combination of the first and second agents. Examples include a glucocorticoid, precursor, analog or glucocorticoid receptor agonist or antagonist. For example, prednisone may be administered, most preferably in the dosage range of about 5-60 milligrams per day. Also, methyl prednisone (Solumedrol) may be administered, most preferably in the dosage range of about 1-2 milligrams per day. Glucocorticoids are currently used to treat relapse episodes in MS patients, and symptomatic RA within this dosage range.

THERAPEUTICALLY EFFECTIVE DOSAGE OF THE PRIMARY AGENT. A therapeutically effective dose of the primary agent is one sufficient to raise the serum concentration above basal levels, and preferably to produce a biological effect on a positive control tissue, such as the uterus, to reduce ER alpha mediated increases in uterine weight.

In one embodiment, where the primary agent is DPN, the preferable dose is from about 1 to 20 milligrams per kilogram daily, and more specifically, about 5-10 milligrams per kilogram daily, or about 8 milligrams per kilogram daily.

The dosage of the primary agent may be selected for an individual patient depending upon the route of administration, severity of disease, age and weight of the patient, other medications the patient is taking and other factors normally considered by the attending physician, when determining the individual regimen and dosage level as the most appropriate for a particular patient.

The use of this group of primary agents is advantageous in at least that other known or experimental treatments for cellular mediated autoimmune diseases are chemotherapeutic immunosuppresants which have significant risks and side effects to patients, including decreasing the ability of the patient to fight infections, inducing liver or heart toxicity which are not caused by estrogen treatment. Other agents used in MS do not cause these side effects, but are associated with flu-like symptoms or chest tightness. Further, these previously used agents are associated with local skin reactions since they entail injections at frequencies ranging from daily to once per week.

DOSAGE FORM. The therapeutically effective dose of the primary agent included in the dosage form is selected at least by considering the primary agent selected and the mode of administration, preferably oral. The dosage form may include the active primary agent in combination with other inert ingredients, including adjutants and pharmaceutically acceptable carriers for the facilitation of dosage to the patient as known to those skilled in the pharmaceutical arts. The dosage form may be any form suitable to cause the primary agent to enter into the tissues of the patient.

In one embodiment, the dosage form of the primary agent is an oral preparation (liquid, tablet, capsule, caplet or the like) which when consumed results in elevated levels of the primary agent in blood serum. The oral preparation may comprise conventional carriers including dilutents, binders, time release agents, lubricants and disintigrants.

Possible oral administration forms are all the forms known from the prior art such as, tablets, dragees, pills or capsules, which are produced using conventional adjuvants and carrier substances. In the case of oral administration it has provided appropriate to place the daily units, which in case comprise a combination of the primary and secondary agents, in a spatially separated and individually removable manner in a packaging unit, so that it is easy to check whether the typically daily taken, oral administration form has in fact been taken as it is important to ensure that there are no taking-free days.

In other embodiments of the invention, the dosage form may be provided in a topical preparation (lotion, crème, ointment, patch or the like) for transdermal application. Alternatively, the dosage form may be provided in a suppository or the like for intravaginal or transrectal application. Alternatively, the agents may be provided in a form for injection or for implantation.

In the transdermal administration of the combination according to the invention, the agents may be applied to a plaster or also can be applied by transdermal, therapeutic systems and are consequently supplied to the organism. For example, an already prepared combination of the agents or the latter individually can be introduced into such a system, which is based on ionotherapy or diffusion or optionally a combination of these effects.

That the agents can be delivered via these dosage forms is advantageous in that currently available therapies, for MS for example, are all injectables which are inconvenient for the user and lead to decreased patient compliance with the treatment. Non-injectable dosage forms are further advantageous over current injectable treatments which often cause side effects in patients including flu-like symptoms (particularly, β interferon) and injection site reactions which may lead to lipotrophy (particularly, glatiramer acetate copolymer-1).

However, in additional embodiment, the dosage form may also allow for preparations to be applied subcutaneously, intravenously, intramuscularly or via the respiratory system.

DOSE AND PREFERRED EMBODIMENTS. By way of example, which is consistent with the current therapeutic uses for these treatments, Avonex® in a dosage of about 0 to about 30 mcg may be injected intramuscularly once a week. Betaseron® in a dosage of about 0 to about 0.25 mg may be injected subcutaneously every other day. Copaxone® in a dosage of about 0 to about 20 mg may be injected subcutaneously every day. Finally, Rebiff® may be injected at a therapeutic dose and at an interval to be determined based on clinical trial data. One objective would be to select the minimal effective dose of β-interferon given the side effects, injection site reactions and compliance issues associated with its use. Thus, the second agent may be administered at a reduced dose or with reduced frequency due to synergistic effects with the primary agent. However, dosages and method of administration may be altered to maximize the effect of these therapies in conjunction with estrogen β receptor ligand treatment. Dosages may be altered using criteria that are known to those skilled in the art of diagnosing and treating autoimmune diseases.

Materials and Methods.

Animals. Female wild type C57BL/6 mice, and ERβ homozygous KO mice on the C57BL/6 background, age 8 weeks, were obtained from Taconic (Germantown, N.Y.). Animals were maintained in accordance with guidelines set by the National Institutes of Health and as mandated by the University of California Los Angeles Office for the Protection of Research Subjects and the Chancellor's Animal Research Committee.

Reagents. Propyl pyrazole triol (PPT) and Diarylpropionitrile (DPN), an ERα and an ERβ agonist, respectively, were purchased from Tocris Bioscience (Ellisville, Mo.). Estradiol was purchased from Sigma-Aldrich (St. Louis, Mo.). Miglyol 812 N liquid oil was obtained from Sasol North America (Houston, Tex.). Myelin oligodendrocytes glycoprotein (MOG) peptide, amino acids 35-55, was synthesized to >98% purity by Mimotopes (Clayton, Victoria, Australia).

Hormone manipulations during EAE. Ovariectomized mice were treated with daily subcutaneous injections of estradiol 0.04 mg/kg/day, DPN at 8 mg/kg/day, PPT at 10 mg/kg/day, or vehicle beginning seven days prior to EAE immunization and throughout the entire disease duration. Vehicle alone treatments consisted of 10% ethanol and 90% migylol. Uterine weights to assess the biological response to dosing were as assessed. Uterine weight was used as a positive control to assess dosing of estrogen receptor agonists. Daily subcutaneous injections of vehicle, estradiol, PPT, or DPN, as well as a combination of PPT with DPN, were administered for ten days at indicated doses to ovariectomized mice. Following euthanasia, the uterus was extracted, then fat, connective tissue, and excess fluid removed in order to obtain the uterine weight, as described

EAE Induction. Active EAE was induced by immunizing with 300 μg of MOG peptide. Some mice were followed clinically for up to 50 days after disease induction, while others were sacrificed earlier for mechanistic studies at day 19 after disease induction, corresponding to day 4-6 after the onset of clinical signs in the vehicle treated group. In some instances, active EAE was induced by immunizing with 300 μg of myelin oligodendrocyte glycoprotein (MOG) peptide, amino acids 35-55, and 500 μg of Mycobacterium tuberculosis in complete Freund's adjuvant. Mice were monitored and scored daily for clinical disease severity according to the standard 0-5 EAE grading scale: 0, unaffected; 1, tail limpness; 2, failure to right upon attempt to roll over; 3, partial paralysis; 4, complete paralysis; and 5, moribund. On each day, the mean of the clinical scores of all mice within a given treatment group were determined, thereby yielding the mean clinical score for that treatment group

Rotarod Testing. Motor behavior was tested up to two times per week for each mouse using a rotarod apparatus (Med Associates Inc, St. Albans, Vt.). Briefly, animals were placed on a rotating horizontal cylinder for a maximum of 200 seconds. The amount of time the mouse remained walking on the cylinder, without falling, was recorded. Each mouse was tested on a speed of 3-30 rpm and given three trials for any given day. The three trials were averaged to report a single value for an individual mouse, and then averages were calculated for all animals within a given treatment group. The first two trial days, prior to immunization (day 0), served as practice trials.

Immune Responses. Splenocytes were stimulated with autoantigen at 25 μg/ml, supernatants were collected after 48 and 72 hours, and levels of TNFα, IFNγ, IL6, and IL5 were determined by cytometric bead array (BD Biosciences).

Histologic Preparation and Immunohistochemistry. Mice were deeply anesthetized with isoflurane and perfused transcardially with ice-cold 0.9% saline, followed by 10% formalin. Spinal cord columns were removed and post-fixed overnight in 10% formalin and eryoprotected with 20% sucrose solution in PBS. Spinal cords were removed from the column and cut in 3 parts (cervical, thoracic and lumbar) and embedded in gelatin/sucrose mix. Spinal cord regions in gelatin were further postfixed and stored in 20% sucrose. Free-floating sections (25 μm thick) were cut coronally with a sliding microtome and collected serially in PBS. Serial sections were mounted on slides and stained with hematoxylin & eosin (H&E) or Nissl. Consecutive sections were also examined by immunohistochemistry. Briefly, 25 μm free-floating sections were permeabilized in 0.3% Triton X-100 in PBS and blocked with 10% normal goat serum. White matter immunostaining was enhanced by treating sections with 95% ethanol/5% acetic acid for 15 minutes prior to permeabilization and blocking. To detect specific cell types and structures, sections were pre-incubated with primary antibodies in PBS solution containing 2% NGS for 2 hours at room temperature, then overnight at 4° C. The following primary antibodies were used: anti-β3 tubulin and anti-neurofilament-NF200 (monoclonal, Chemicon; polyclonal Sigma Biochemical), anti-neuronal specific nuclear protein (NeuN), anti-CD45 (Chemicon), and anti-MBP (Chemicon). The second antibody step was performed by labeling with antibodies conjugated to TRITC, FITC and Cy5 (Vector Labs and Chemicon). IgG-control experiments were performed for all primary antibodies, and no staining was observed under these conditions. To assess the number of cells, a nuclear stain 4′,6-Diamidino-2-phenylindole, DAPI (2 ng/ml; Molecular Probes) was added for 15 minutes prior to final washes after secondary antibody addition. The sections were mounted on slides, dried and coverslipped in fluoromount G (Fisher Scientific).

Microscopy and Quantification. Sections from spinal cord levels T1-T5 were examined, six from each mouse, with n=3 mice per treatment group, for a total of 18 sections per treatment group. Images were captured under microscope (4×, 10× or 40×) using the DP70 Image software and a DP70 camera (both from Olympus). All images were converted to grayscale and then analyzed by density measurement with ImageJ v1.29. Increase in total number of infiltrating cells was measured by density measurements of DAPI⁺ nuclei in the whole white matter. Neuronal cells were quantified by counting the NeuN⁺/(β3-tubulin⁺/DAPI⁺ cells per mm² in the whole gray matter. Laser scanning confocal microscopic scans were performed on MBP⁺/NF200⁺ and CD45⁺/NF200⁺ immunostained spinal cord sections, each as described.

Statistical Analysis. EAE clinical disease severity was compared between treatment groups using the Friedman test; histopathological changes were assessed using 1×4 ANOVAs; uterine weights, proliferative responses and cytokine levels were compared between treatment groups using Student t-test, and time on rotorod was compared between treatment groups using ANOVA.

Results

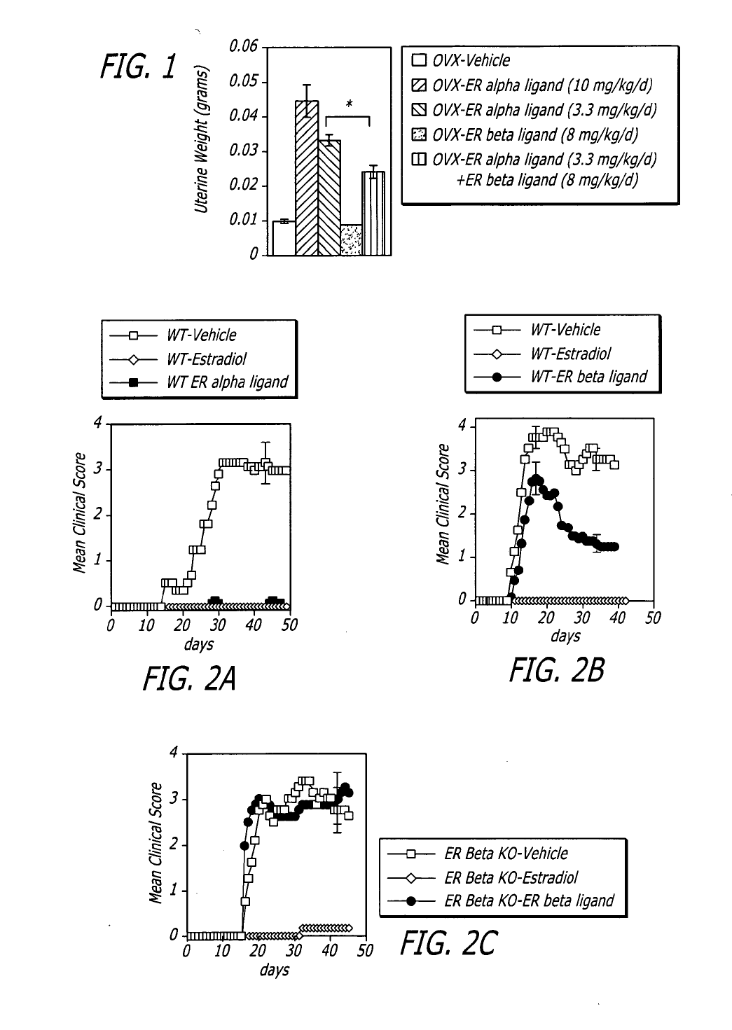

Selected doses of ERα and ERβ ligands induced known biological responses on a positive control tissue, the uterus. Before beginning EAE experiments, the uterine response was used to assess whether a known in vivo response would occur during treatment with each of dosing regimen for the ERα and ERβ ligands. It was known that estrogen treatment increased uterine weight primarily though ERα, and it had also been shown that treatment with the ERβ ligand diarylpropionitrile (“DPN”) could antagonize the ERα mediated increase in uterine weight. Thus, the ERα ligand, propyl pyrazole triol (“PPT”), was administered to ovariectomized C57BL/6 females for 10 days at either an optimal (10 mg/kg/day) or suboptimal (3.3 mg/kg/day) dose, and observed a significant increase in uterine weight as compared to vehicle treated (FIG. 1). When an ERβ ligand dose (8 mg/kg/day) was given in combination with the ERα ligand, the increase in uterine weight mediated by ERα ligand treatment was significantly reduced. These data demonstrated that the method and dose of delivery of the ERα and ERβ ligands induced a known biological response in vivo on a positive control tissue, the uterus.

Differential effects of treatment with ERα and ERβ ligands on clinical EAE. Effects between ERα and ERβ treatment were compared and contrasted during EAE. When the ERα ligand was administered one week prior to active EAE induction with MOG 35-55 peptide in ovariectomized C57BL/6 female mice, clinical disease was completely abrogated, p<0.0001 (FIG. 2A). This was consistent with previous findings in this EAE model, as well as findings in adoptive EAE in SJL mice by others. In contrast, ERβ ligand treatment had no significant effect early in disease (up to day 20 after disease induction), but then demonstrated a significant protective effect later in disease (after day 20), p<0.001 (FIG. 2B).

Data showing a protective effect using the ERβ ligand DPN in active EAE in C57BL/6 mice were surprising given that: 1) another ERβ ligand (WAY-202041) was shown to have no effect in EAE, albeit using a different strain of mice which were followed for a shorter time period and 2) WAY-202041 was shown to have a 200 fold selectivity for ERβ, while DPN has a 70 fold selectivity. To assess the in vivo selectivity of DPN treatment during EAE, DPN was administered to homozygous ERβ knock out (KO) mice. When DPN was administered to ovariectomized ERβ KO C57BL/6 mice with EAE, the treatment was no longer protective (FIG. 2C). These data demonstrated the in vivo selectivity of DPN for ERβ during EAE at the dose used in the studies.

Together these results indicated that treatment with an ERα ligand is protective at the acute onset and throughout the course of EAE, while treatment with an ERβ ligand is protective during the later phase of the disease, after the acute initial phase.

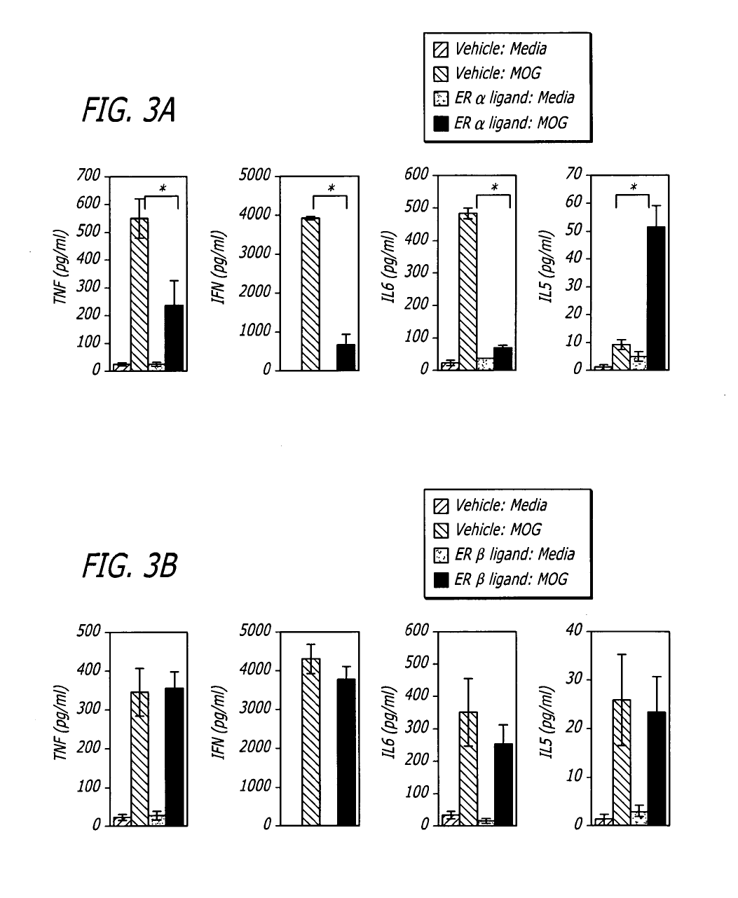

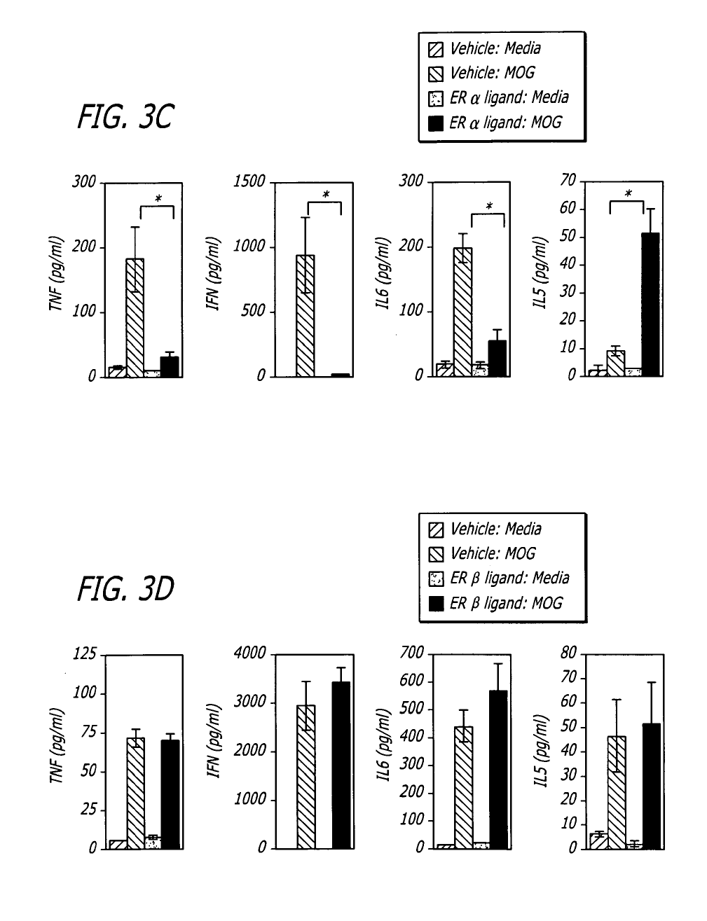

Differential effects of treatment with ERα and ERβ ligands on autoantigen specific cytokine production. To further investigate differences between treatments with the ERα versus the ERβ ligand, autoantigen specific cytokine production by systemic immune cells during EAE was assessed. ERα ligand treatment significantly reduced levels of cytokines (TNFα, IFNγ, and IL6) known to be pro-inflammatory in EAE, while it increased the anti-inflammatory cytokine IL5, during both early (FIG. 3A) and later (FIG. 3C) stages of EAE. In contrast, ERβ ligand treatment was no different from vehicle treatment in all measured cytokines (TNFα, IFNγ, IL6, and IL5) at either the early (FIG. 2B) or later (FIG. 3D) time points. These results indicated that while ERα ligand treatment was anti-inflammatory in the systemic immune system, ERβ ligand treatment was not.

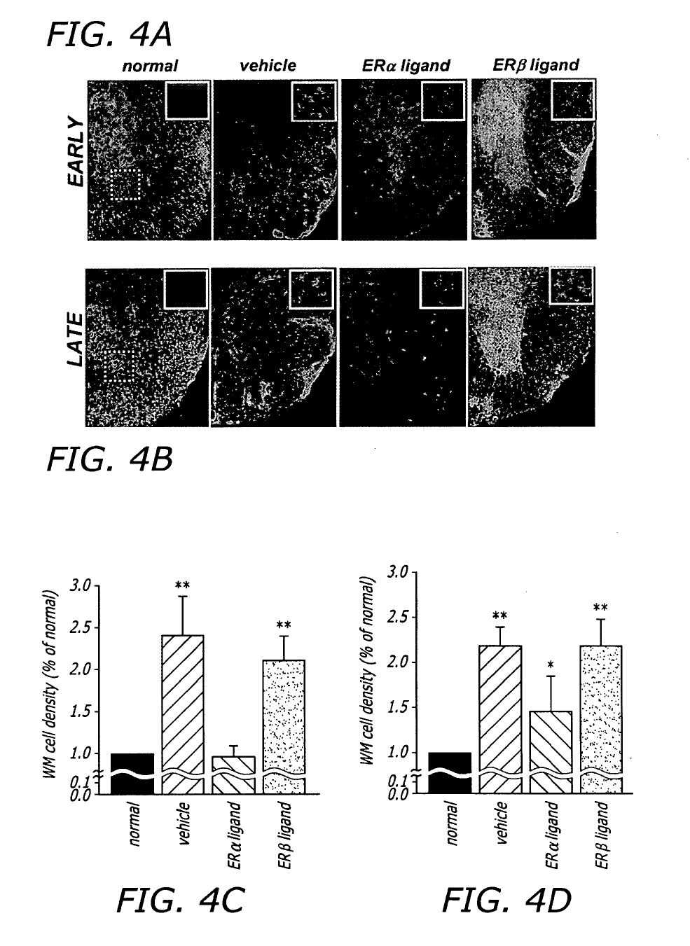

Treatment with an ERα ligand, but not an ERβ ligand, reduces CNS inflammation. Whether treatment with ERα versus ERβ ligands resulted in differences in inflammation within the CNS, was addresses. At both early (day 19) and later (day 40) stages of EAE, spinal cord sections from mice treated with either vehicle, ERα or ERβ ligand were assessed for inflammation using anti-CD45 antibody to stain inflammatory cells. ERα ligand treated EAE mice, compared to vehicle treated EAE, had less CD45 staining in white matter. This reduction in CD45 staining was present at both the early (FIG. 4A) and later (FIG. 4B) timepoints in EAE. In contrast, ERβ ligand treated EAE mice did not have reduced CD45 staining in white matter, at either time point. Additionally, CD45 staining of cells in gray matter of vehicle treated EAE mice was observed at both the early and later time points, with these cells demonstrating a morphology suggestive of activated microglia (FIG. 4 insets). This gray matter inflammation was also decreased with ERα ligand, but not ERβ ligand, treatment.

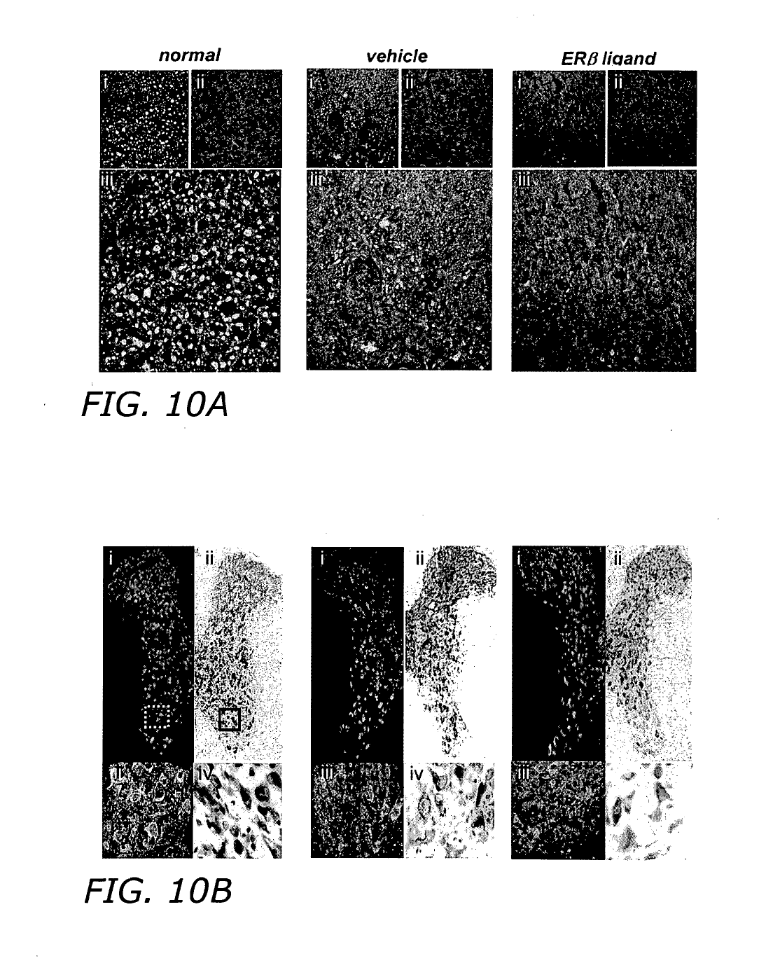

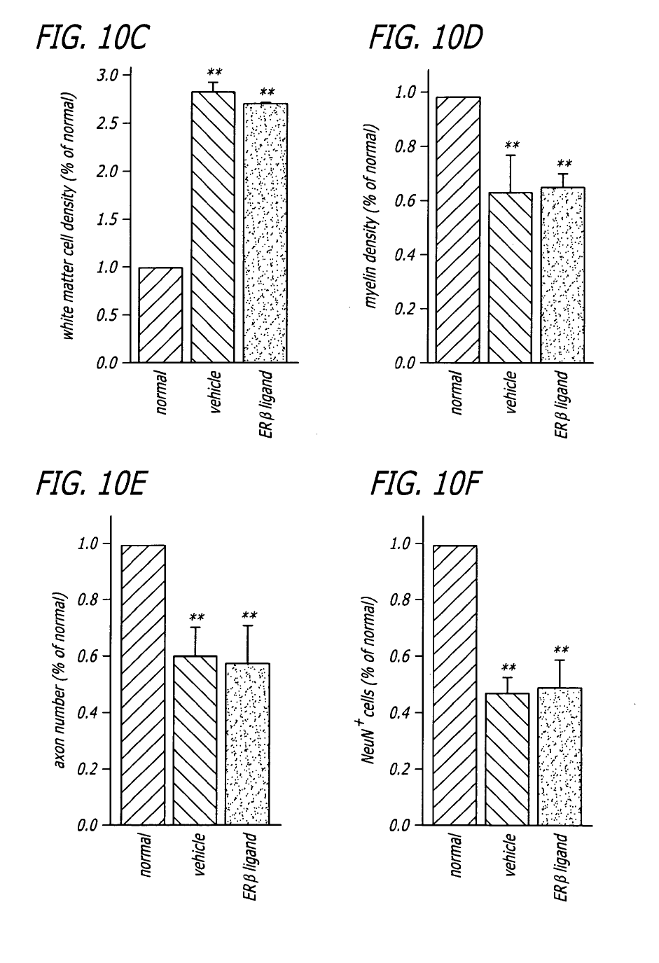

Hemotoxylin and eosin (H&E) staining also revealed that vehicle treated EAE mice had extensive white matter inflammation at both the early (FIG. 10A) and later (FIG. 10B) time points. This inflammation was reduced by treatment with the ERα, but not the ERβ ligand. Quantification of white matter cell density revealed that ERα ligand treated mice at the early stage of EAE had a reduction in inflammation, such that levels were no different as compared with those in normal control mice, while white matter cell densities in ERβ ligand treated EAE mice remained significantly increased and comparable to those in vehicle treated EAE mice, FIG. 4C. At the later time point, quantification detected some inflammation in ERα ligand treated EAE mice, while inflammation in ERβ ligand treated remained very high and similar to vehicle treated EAE mice (FIG. 4D).

Together these data indicated that ERα ligand treatment, but not ERβ ligand treatment, reduced inflammation in the CNS of mice with EAE.

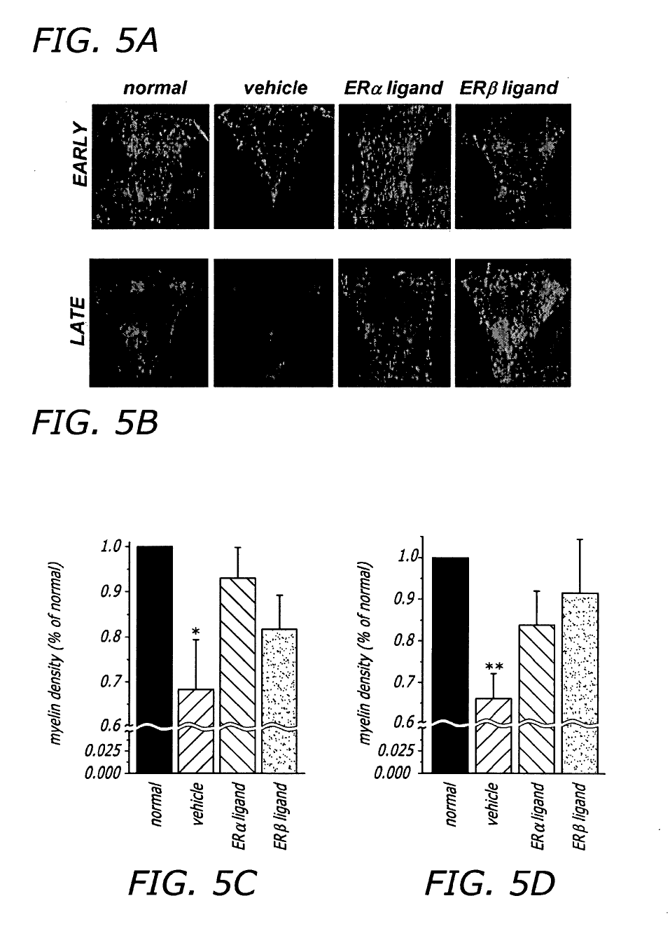

Treatment with both an ERα ligand and an ERβ ligand reduces demyelination in white matter. The degree of myelin loss was then assessed by myelin basic protein (MBP) immunostaining in the dorsal columns of thoracic cords. Extensive demyelination occurred at the sites of inflammatory cell infiltrates in vehicle treated EAE mice while less demyelination occurred in ERα and ERβ ligand treated (FIG. 5A, B). Quantification of demyelination by density analysis of MBP immunostained spinal cord sections revealed a 32% (p<0.01) and 34% (p<0.005) decrease in myelin density in vehicle treated EAE mice, at the early and later time points, respectively, as compared to healthy controls (FIG. 5C, D). In contrast, myelin staining was somewhat decreased, but relatively preserved in both ERα and ERβ ligand treated mice, with no significant difference as compared to healthy controls.

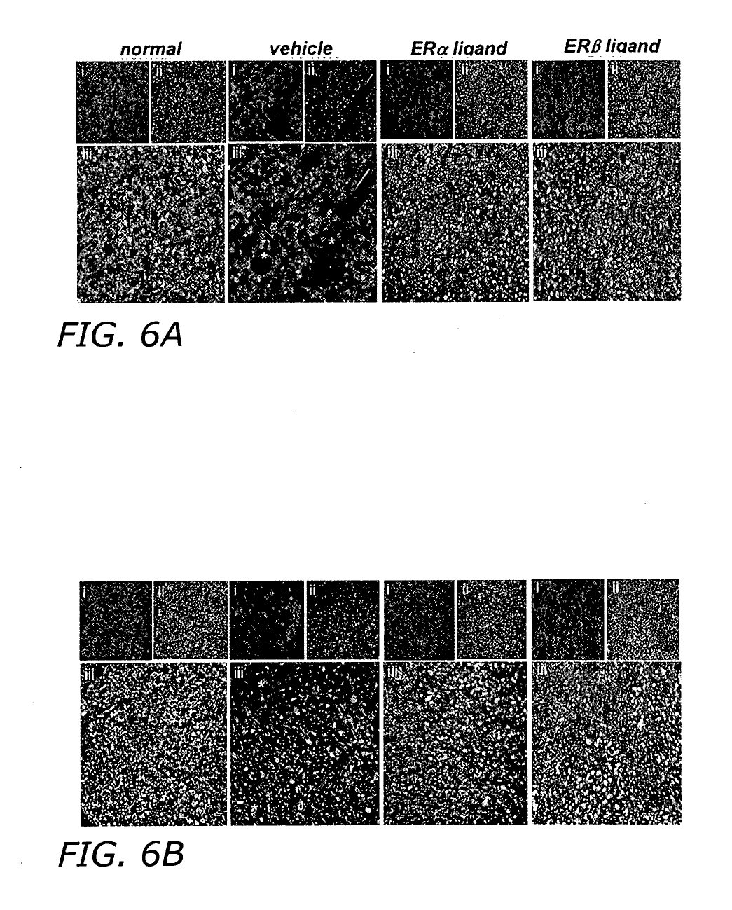

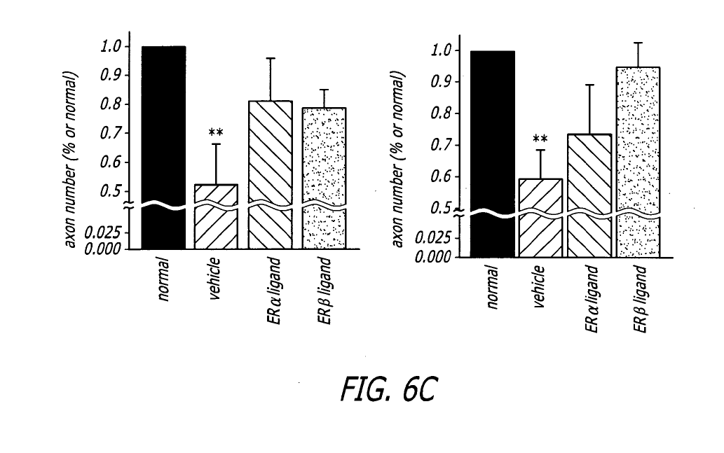

Treatment with both an ERα ligand and an ERβ ligand reduces axonal loss in white matter. Staining with anti-NF200 antibody revealed axonal loss in white matter of vehicle treated EAE mice at both early and later time points of disease as compared to healthy controls, while both ERα ligand and ERβ ligand treated had less axonal loss (FIG. 6A, B). Quantification of NF200 staining in anterior fununculus revealed a 49±12% (p<0.01) and 40±8% (p<0.005) reduction in vehicle treated EAE, at the early and later time points, respectively, as compared to healthy controls (FIG. 6C, D), while axon numbers in ERα ligand and ERβ ligand treated EAE mice were not significantly reduced as compared to those in healthy controls.

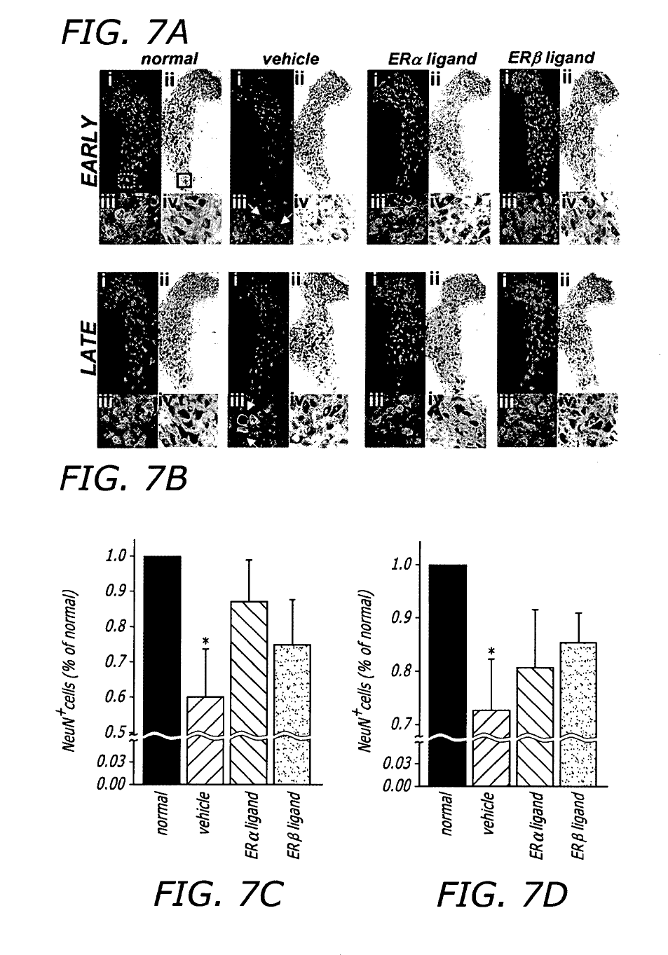

Treatment with both an ERα ligand and an ERβ ligand reduces neuronal pathology in gray matter. A recent report demonstrated neuronal abnormalities surprisingly early during EAE (day 15), which were prevented by treatment with either estradiol or ERα ligand. Here, it was asked whether ERβ ligand treatment might preserve neuronal integrity. A combination of Nissl stain histology and NeuN/β3-tubulin immunolabeling was used to identify and semi-quantify neurons in gray matter. A decrease in neuronal staining in gray matter occurred at both time points in vehicle treated EAE mice as compared to healthy controls, while neuronal staining in gray matter was preserved in EAE mice treated with either the ERα or the ERβ ligand at the early and the later time points (FIG. 7A, B). Quantification of NeuN⁺ cells in gray matter demonstrated a 41±13% (p<0.05) and 31±8% (p<0.05) reduction, at the early and later time points respectively, in vehicle treated EAE mice as compared to normal controls, while ERα and ERβ ligand treated mice had NeuN⁺ cell numbers that were fewer, but not significantly different from those in healthy controls (FIG. 7C, D).

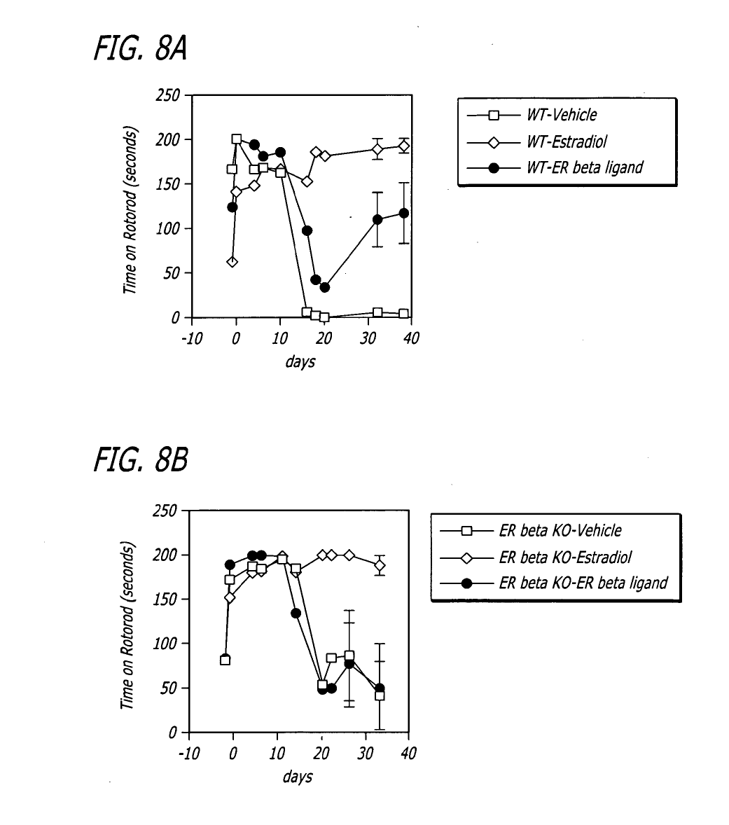

Treatment with an ERβ ligand induces recovery of motor performance. Since treatment with an ERβ ligand was found to be neuroprotective in EAE, the clinical significance of this neuroprotective effect using an outcome frequently used in spinal cord injury, rotarod performance was assessed. Vehicle treated EAE mice demonstrated an abrupt and consistent decrease in the number of seconds they were able to remain on the rotarod, beginning at day 12 after disease induction, and this disability remained throughout the observation period. In contrast, ERβ ligand treated mice had an abrupt decrease in the number of seconds they could remain on the rotarod apparatus, but later, at days 30-40, they had significant recovery (FIG. 8A). These data demonstrated that ERβ ligand treatment induced functional clinical recovery in motor performance at later time points of disease during EAE. Finally, the improvement in rotarod performance with DPN treatment was no longer observed in the ERβ KO (FIG. 8B), demonstrating that the DPN induced recovery in motor performance later in disease was indeed mediated through ERβ.

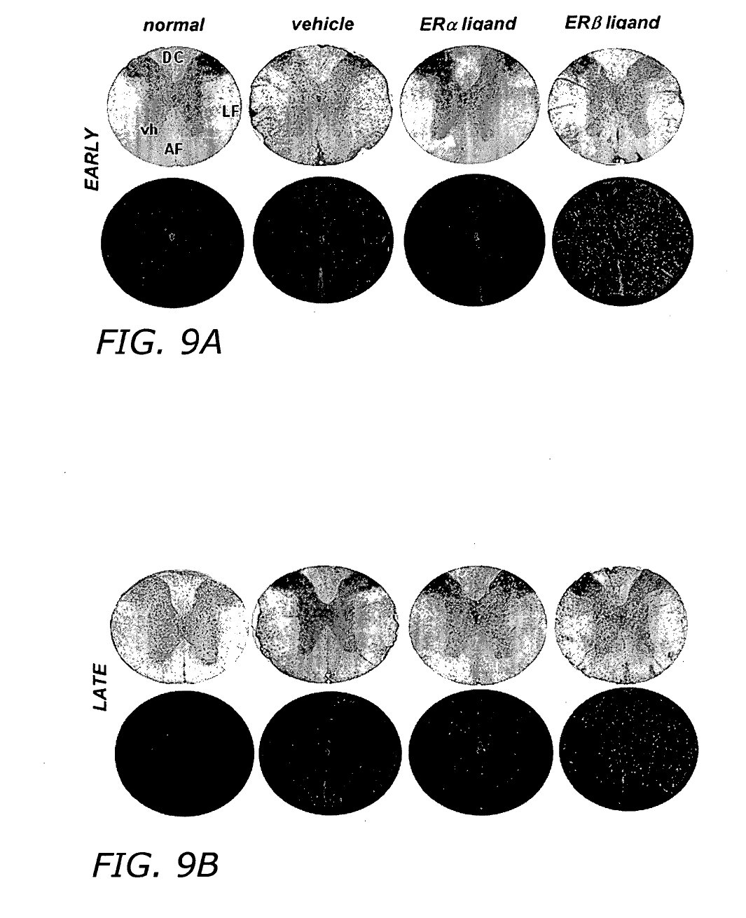

Treatment with an ERα ligand, not an ERβ ligand, reduced inflammation in spinal cords of mice with EAE: H&E and DAPI staining. FIG. 9(A) Representative H&E (top) and DAPI (bottom) stained thoracic spinal cord sections (4× magnification) from healthy control, as well as vehicle, ERα ligand (PPT) and ERβ ligand (DPN) treated EAE mice, all sacrificed at day 19 (early) post-disease induction. Compared to controls, vehicle treated EAE spinal cords showed multifocal to coalescing areas of inflammation in the leptomeninges and white matter, around blood vessels, and in the parenchyma of the white matter. (DC—dorsal column; LF—lateral funiculus; vh—ventral horn; AF—anterior funnicilus). ERα ligand treated spinal cords had reduced inflammation as compared to vehicle treated EAE, while ERβ ligand did not have reduced levels of inflammation. (B) Day 40 (late) after disease induction, as in (A) above (FIG. 9).

Protection from neuropathology is mediated by ERβ. To confirm whether the effect of DPN treatment in vivo on CNS neuropathology was indeed mediated through ERβ, neuropathology in DPN treated EAE mice deficient in ERβ was assessed. At day 38 after disease induction, inflammation, demyelination and reductions in axon numbers were present in white matter, while neuronal staining was decreased in gray matter of vehicle treated EAE mice (FIG. 10). In contrast to the neuroprotection observed during DPN treatment of wild type mice (FIG. 5-7), DPN treatment of ERβ knock out mice failed to prevent this white and gray matter pathology (FIG. 10). These data demonstrated that neuroprotective effects mediated by DPN treatment in vivo during EAE are mediated through ERβ.

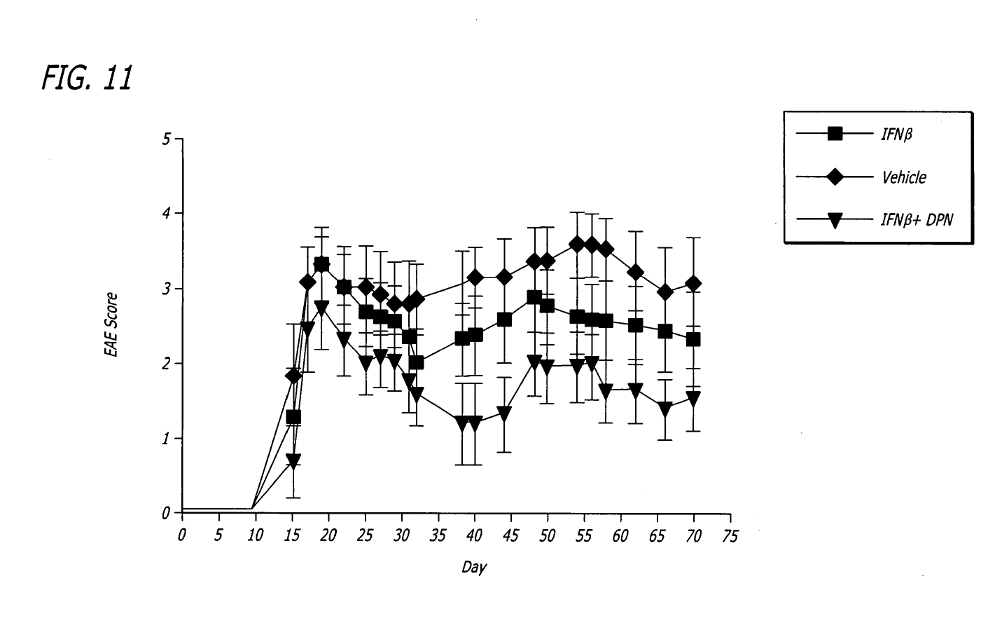

Combination treatment with IFNβ and an estrogen receptor beta (ERβ) ligand reduce the severity of disability in experimental autoimmune encephalomyelitis (EAE). Eight-week old female C57BL/6 mice were treated with placebo vehicle (n=7, black), IFNβ (n=8, red) at a dose of 20 KU as described, or with the combination of IFNβ 20 KU combined with the ERβ ligand DPN (n=8, green) at a dose of 8 mg/kg/day, as described. EAE was induced with MOG 35-55 peptide as described. Animals were monitored daily for EAE signs based on a standard EAE 0-5 scale scoring system: 0—healthy, 1—complete loss of tail tonicity, 2—loss of righting reflex, 3—partial paralysis, 4—complete paralysis of one or both hind limbs, and 5—moribund. IFNβ treatment alone led to mild reduction in chronic EAE severity when compared to vehicle treated animals from day 30 to endpoint day 70. In contrast, combination treatment of IFNβ and DPN was superior, leading to greater reduction in EAE disease severity as compared to treatment with IFNβ alone or vehicle (FIG. 11).

This suggests a synergistic effect of IFNβ (an anti-inflammatory) treatment for MS and other autoimmune diseases and ERβ agonist (which has been demonstrated to be neuroprotective in EAE, the animal model for MS).

This synergistic effect is advantageous in that the combination provides an added benefit in clinical score overall in the EAE model. The benefit to patients may be to reduce the dose of traditional IFNβ therapies, which have negative side effects and low compliance. Further, it would be an advantage to treat MS with not only a anti-inflammatory agent (as is the case with all approved drugs) but also with a neuroprotective agent (none currently available). This is because it is known that brain atrophy and clinical disability in MS continue despite good anti-inflammatory treatments probably because while the inflammation may start the disease, it soon takes on a neurodegenerative component. This neurodegenerative component of the disease continues, becoming independent of the inflammatory component. Thus when one blocks only inflammation and does not stop neurodegeneration, brain atrophy and clinical disability continues. The possibility would be that combined treatment with an a standard anti-inflammatory agent and a novel neuroprotective agent would halt, not merely slow, disability accumulation. Thus, the provision of neuroprotective therapy is advantageous in decreasing symptomology, increasing overall clinical score and disability in MS patients.

Although the present invention has been described in terms of the preferred embodiment above, numerous modifications and/or additions to the above-described preferred embodiments would be readily apparent to one skilled in the art.

What is claimed is:

1. A method for treating the symptoms of a neurodegenerative disease in a mammal, the method comprising the steps of administering to the mammal a primary agent being an estrogen receptor ligand and a secondary agent being an immunotherapeutic compound.

2. The method of claim 1, wherein the estrogen receptor ligand is an estrogen receptor beta ligand.

3. The method of claim 1, wherein the secondary agent is beta-interferon.

4. The method of claim 3, wherein the beta-interferon is interferon-β 1a or interferon-β 1b.

5. The method of claim 1, wherein the neurodegenerative disease is selected from the group comprising: multiple sclerosis, Alzheimer's disease, or Parkinson's disease.

6. The method of claim 1, wherein the symptoms of the neurodegenerative disease are treated using a compound comprising a primary agent being an estrogen receptor ligand and a secondary agent being an immunotherapeutic compound.

7. The method of claim 7, wherein the estrogen receptor ligand is an estrogen receptor beta ligand.

8. The method of claim 7, wherein the secondary agent is beta-interferon.

9. A method to treat the symptoms of multiple sclerosis, comprising the steps of administering a primary agent estrogen receptor ligand and a secondary agent being an immunotherapeutic compound.

10. The method of claim 9, wherein the primary agent estrogen receptor ligand is an estrogen receptor beta ligand.

11. The method of claim 10, wherein the secondary agent immunotherapeutic compound is a beta interferon.

12. A compound agent for use in the treatment of the symptoms of a neurodegenerative disease, the compound comprising a primary agent being an estrogen receptor ligand and a secondary agent being an immunotherapeutic compound.

13. The compound of claim 12, wherein the primary agent estrogen receptor ligand is an estrogen receptor beta ligand.

14. The compound of claim 12, wherein the secondary agent immunotherapeutic compound is a beta interferon.

15. A compound agent for use in the treatment of the symptoms of a neurodegenerative disease, the compound comprising a primary agent being an estrogen receptor ligand and a secondary agent being an immunotherapeutic compound, whereby administration of the compound reduces demyelination in white matter.

16. The compound of claim 15, wherein the primary agent estrogen receptor ligand is an estrogen receptor beta ligand.

17. The compound of claim 15, wherein the secondary agent immunotherapeutic compound is a beta interferon.

18. A compound agent for use in the treatment of the symptoms of a neurodegenerative disease, the compound comprising a primary agent being an estrogen receptor ligand and a secondary agent being an immunotherapeutic compound, whereby administration of the compound reduces axonal loss in white matter.

19. The compound of claim 18, wherein the primary agent estrogen receptor ligand is an estrogen receptor beta ligand.

20. The compound of claim 18, wherein the secondary agent immunotherapeutic compound is a beta interferon.

21. A compound agent for use in the treatment of the symptoms of a neurodegenerative disease, the compound comprising a primary agent being an estrogen receptor ligand and a secondary agent being an immunotherapeutic compound, whereby administration of the compound reduces neuronal pathology in gray matter.

22. The compound of claim 21, wherein the primary agent estrogen receptor ligand is an estrogen receptor beta ligand.

23. The compound of claim 21, wherein the secondary agent immunotherapeutic compound is a beta interferon.

24. A compound agent for use in the treatment of the symptoms of a neurodegenerative disease, the compound comprising a primary agent being an estrogen receptor ligand and a secondary agent being an immunotherapeutic compound, whereby administration of the compound induces recovery of motor performance.

25. The compound of claim 24, wherein the primary agent estrogen receptor ligand is an estrogen receptor beta ligand.

26. The compound of claim 24, wherein the secondary agent immunotherapeutic compound is a beta interferon.

27. Use of at least one primary therapeutically active agent, the primary therapeutically active agent being an estrogen receptor ligand, in combination with a secondary active agent, the secondary active agent being beta interferon for the manufacture of a medicament for the therapeutic treatment of a neurodegenerative disease in a mammal.

28. The use as claimed in claim 27, wherein the primary therapeutically active agent is an estrogen receptor beta ligand.
